(19) United States
(12) Patent Application Publication
(10) Pub. No.: US 2006/0160144 A1
(43) Pub. Date: Jul. 20, 2006

(54) Sensor arrangement, device and method for testing active substances and/or active sites from a pharmacological point of view using an amperometer and/or potentiometer

(75) Inventor: Klaus Fendler, Rodgau-Niederroden (DE)

Correspondence Address: ARENT FOX PLLC, 1050 CONNECTICUT AVENUE, N.W., SUITE 400, WASHINGTON, DC, 20036, US

(73) Assignee: IONGATE BIOSCIENCES GMBH

(21) Appl. No.: 11/384,480

(22) Filed: Mar. 21, 2006

Related U.S. Application Data
(62) Division of application No. 10/469,707, filed on Feb. 4, 2004.

(30) Foreign Application Priority Data
Mar. 15, 2001 (DE) 101 12 505.4

(51) Int. Cl. G01N 33/53; C12M 1/34

(52) U.S. Cl. 435/7.1; 435/287.2; 205/777.5

(57) ABSTRACT

The invention relates to a sensor electrode device (20) for carrying out amperometric and/or potentiometric testing of active sites or substances from a pharmacological point of view. In order to carry out testing in a reliable, quick and inexpensive manner, the device is provided with a solid-supported electrode area (21) and is electrically insulated in relation to the measuring medium (30) and primary supports (10) mounted on the electrode area. Vesicules and membrane fragments, inter alia, are used as primary supports.

Sensor arrangement and device for pharmacological testing of an active site and/or active ingredient and process for pharmacological testing of an active site and/or active ingredient using amperometric and/or potentiometric means.

The invention relates to a sensor arrangement and device for pharmacological testing of an active site and/or active ingredient using amperometric and/or potentiometric means and a process for pharmacological testing of an active site and/or active ingredient using amperometric and/or potentiometric means.

Before semi-luxury foods, medications and other preparations can be used in man, plants and animals, certain criteria with respect to the demonstration of their effectiveness and/or safety, therefore with respect to their side effects, must be satisfied relative to approval for the market. Accordingly, in the prior art different processes and measurement means are known with which the corresponding properties of certain active ingredients can be quantitatively and qualitatively analyzed and described.

On the other hand, it is often desirable to apply given and known active ingredients to a certain active site, for example to a certain protein, to a cell, a tissue or the like, in order to influence and/or study the manner of operation of this active site.

Conventional experiments on the overall organism are often objectionable due to the complexity of the organism and furthermore based on ethical and moral circumstances, and the results which have been achieved with them are meaningful only to a limited degree.

Consequently processes and means have been developed using which isolated examination of how the active ingredients which are to be tested act on isolated action centers or a study of the action centers themselves—especially within the framework of a deorphaning process for an unknown function of the action center—is possible. In doing so certain tissues, isolated cells and/or other biological units, for example proteins or the like, are made accessible to examination in an isolated manner. For example, techniques are known which work using stains and which use and represents changing bonding specificities. If this procedure is possible at all, it is however disadvantageous with respect to the fact that it has low resolution capacity and poor sensitivity, but high fault susceptibility.

On the other hand, however, there are electrophysiological methods, for example the so-called patchclamp technique or voltage clamp technique, in which for example cells are accessible in isolation to electrophysiological examination. In this process native cells are subjected to different conditions and certain electrical activities which are imparted by the cells via the organelles and/or proteins or the like which are contained in them are measured. In spite of the high precision which can be achieved in this, these known electrophysiological methods are disadvantageous in that they often yield only little reproducibility of the results; due to their high measurement engineering complexity and their fault susceptibility they are unsuited to prompt, automated and/or broad use and have certain problems in the discrimination of unwanted signal portions due to the generally native environment of the objects which are to be studied.

DE 190 07 279 A1 discloses solid-supported membrane biosensors. These known membrane biosensors consist of a solid as the carrier, a lipid double layer as the membrane and a spacer which is installed between the carrier and the lipid double layer. The lipid double layer has an intercalated receptor as the biological unit. In the area of the spacer, therefore between the lipid double layer and the solid carrier and on the side of the lipid double layer facing away from the solid there is an aqueous measurement medium, by which a natural environment for maintaining their biological function is imparted to the installed receptors.

Another sensor form is disclosed in DE 44 37 274 A1. There is a solid carrier there on which a layer specific to the analyzed substance consisting of a liquid, solid or semisolid material is applied. On the carrier itself there are optionally several electrodes which are embedded in the layer specific to the analyzed substance in contact with the carrier. The layer specific to the analyzed substance is a layer of material into which optionally also polypeptides or proteins, receptors or the like can be embedded as biological units. In this known sensor, between the layer specific to the analyzed substance as the primary carrier and the solid carrier which is regarded as the secondary carrier there can additionally be electrical insulation, for example of teflon.

The object of the invention is to devise a sensor arrangement, device and a process for pharmacological testing of an active site and/or active ingredient or the like using amperometric and/or potentiometric means, in which prompt testing of the active site and/or active ingredient, especially in large-scale operation at reasonable cost, is possible in an especially simple and still reliable manner.

The object is achieved as claimed in the invention in a sensor arrangement by the characterizing features of claim 1. Furthermore the object is achieved in a device as claimed in the invention by the characterizing features of claim 11. Moreover the object is achieved in a process for pharmacological testing of an active site and/or active ingredient using amperometric and/or potentiometric means as claimed in the invention with the characterizing features of claim 14.

Advantageous developments of the sensor arrangement as claimed in the invention, the device and the process are the subject matter of the respective dependent claims.

In the sensor arrangement as claimed in the invention for pharmacological testing of an active site and/or active ingredient using amperometric and/or potentiometric means there is a secondary carrier which has an electrically conductive and solid-like electrode area. Furthermore there is a plurality of primary carriers which are located in the immediate spatial vicinity of the secondary carrier and which have biological units, especially membrane proteins, which can be activated into electrical action. In addition, there is an aqueous measurement medium which contains the primary carriers and the secondary carriers. The electrode area is made electrically insulated relative to the measurement medium, the primary carrier and relative to the biological units. The primary carriers can be a eukaryotic cell, a procaryotic cell, a bacterium, a virus, or components, especially membrane fragments, or associations thereof in native form or in altered form, especially in purified, microbiologically and/or molecular biologically altered form. Alternatively or in addition primary carriers can also be a vesicle, a liposome or a micellar structure.

The decisive component of the sensor arrangement as claimed in the invention is therefore a sensor electrode means. This sensor electrode means for pharmacological testing of an active site and/or active ingredient using amperometric and/or potentiometric means itself therefore has at least one electrically conductive electrode area. The sensor electrode means is made to be located in an aqueous measurement medium in operation. Furthermore, the sensor electrode means is made to place a plurality or host of the primary carriers with biological units which can be activated into electrical action, especially membrane proteins or the like, in the immediate spatial vicinity, especially of the electrode area. Here at least the electrode area as claimed in the invention is made solid-like. Furthermore, as claimed in the invention the electrode area is made to be electrically insulated relative to the measurement medium which is to be provided and relative to the primary carriers.

It is thus a key idea of this invention to make at least the electrode area of the sensor arrangement as claimed in the invention and especially of the sensor electrode means solid-like or solid-supported. In this way the sensor electrode means and especially the intended electrode area thereby acquire especially high mechanical stability, by which especially durable and reliable operation within the framework of testing of the active site and/or the active ingredient is possible.

Only by solid-support does activation for example of membrane proteins by a concentration jump become possible. This can take place especially within the framework of a high-speed and/or continuous solution exchange, by which, especially in amperometric measurements, a high signal level and thus high sensitivity can be achieved. As result of the durability due to solid support easier handling and comfortable installation are also possible.

Another key aspect of this invention consists in making the electrode area such that it is made electrically insulated in operation relative to the measurement medium and relative to the primary carriers. This measure results in that the sensor electrode means can be used for example as a capacitively coupled electrode. With respect to the signal/noise ratio, therefore with respect to the detection accuracy, this has major advantages. Furthermore, in the capacitive coupling of the electrode area of the sensor electrode arrangement or means no chemical reaction is involved, as would be the case for example in a typical electrochemical half cell.

Another key aspect of the sensor arrangement as claimed in the invention is the selection of the primary carriers which carry the biological units. The primary carriers can each be eukaryotic cells, procaryotic cells, bacteria, viruses, or components, especially membrane fragments, or associations thereof, in native form or in modified form, especially in purified, molecular biologically and/or microbiologically altered form. Alternatively or in addition as primary carriers also vesicles, liposomes or micellar structures are conceivable.

In one especially advantageous embodiment of the sensor arrangement as claimed in the invention it is provided that the electrode area has at least one electrically conductive electrode, that there is an electrically insulating insulation area and that the respective electrode is electrically insulated by the insulation area from the measurement medium, from the primary carriers and from the biological units.

The electrode area therefore advantageously has at least one electrode. This can on the one hand be made itself as a mechanically stable material area, especially as a plate, a wire and/or the like.

On the other hand, the electrode area can have a carrier which is made especially solid-like. Then it is possible for the electrode to be made as a material area or material layer on the surface area or the surface of this carrier, especially in a coherent manner. Here it is provided especially that the electrode acquires mechanical stability by the solid support by the carrier. This procedure has the advantage that optionally high quality materials can be applied for example as a thin layer to the carrier so that the possibility of a disposable sensor electrode means which can be produced at reasonable price and can be marketed with respect to business arises. Optionally the carrier, especially the electrode area, can be re-used, and especially a renewed insulation area, for example a new thiol layer, can become necessary.

Preferably the electrode has at least one metallic material or is formed from such a material. Here especially a chemically inert precious metal, preferably gold, becomes advantageous. Platinum or silver is also especially conceivable.

On the other hand it can also be advantageous for the electrode not to be made metallic or not purely metallic and to have for example at least one conductive metal oxide. Indium tin oxide for example is a good choice because it is comparatively insensitive to radiation at least in the visible and in the UV range, at least compared to pure metal electrodes.

The carrier for holding the electrode therefore advantageously has an electrically insulating material or is made of such a material. Furthermore or alternatively it is advantageous for the material of the carrier to be essentially chemically inert. Advantageously the material can be glass or the like. Here the shape can be that of a plate or the like. The chemical inertness prevents both a change of the carrier and also contamination of the measurement medium during the measurement process. Choosing an electrically insulating carrier ensures that all the measurement signals originate essentially from the area of the electrode.

One especially simple arrangement of the sensor electrode means results when the electrode is made essentially as a material layer which has been deposited on the surface of the carrier. The material layer can be vapor deposited or sputtered. The material layer for forming the electrode preferably has a layer thickness of roughly 10 to 200 nm.

Between the material layer for the electrode and the surface of the carrier optionally an adhesive layer can be advantageous. Especially when a gold electrode is applied to glass can an interposed adhesive layer of chromium or the like be advantageous. The adhesive layer advantageously has a comparatively small layer thickness, preferably of roughly 5 nm.

To form the capacitive electrode and the insulation of the electrode area from the measurement medium and/or from the primary carriers, which insulation is necessary for this purpose, therefore at least one insulation area is formed by which at least in the operation of the electrode area, especially the electrode can be essentially electrically insulated, especially in areas of it which in operation are intended for mechanical contact with the measurement medium and/or with the primary carriers.

In another preferred embodiment of the sensor arrangement as claimed in the invention the insulation area is made layer-like. Here the insulation area consists at least in part of a sequence of monolayers, the monolayers being made as spontaneously self-organizing layers.

Here it is advantageous for the underlayer of the insulation area to be a layer of an organic thio compound as the lowermost region of the insulation area facing the electrode, preferably a long-chain alkane thiol, especially an octadecane thiol. Furthermore the upper layer of the insulation area is a layer of an amphiphilic organic compound, especially of a lipid, as the uppermost region or surface of the insulation area facing away from the electrode.

It can therefore be advantageous to make the insulation area at least partially layer-like, especially multilayered. This intensifies the insulation action and simplifies production. In order to obtain a rate of attached and/or positioned primary carriers as high as possible in the area of the sensor electrode means in operation, according to one preferred embodiment of the sensor electrode means as claimed in the invention it is provided that at least the surface region of the insulation area is made matched such that attachment and/or placement of the primary carriers on the surface region of the insulation area is promoted, especially in a manner which is compatible with the surface of the primary carrier. This means that depending on the surface composition of the primary carriers the surface region of the insulation area is made matched accordingly to the sensor electrode means so that the primary carriers are favorably attached on the surface region of the insulation area and also remain there.

With respect to especially pronounced capacitive coupling of the sensor electrode means in measurement operation it is provided that the insulation area is made at least partially as a monomolecular film, monolayer and/or as a sequence of them. In this case the specific electrical capacitance of the electrode boundary layer referenced to the area is especially high. The arrangement and execution of the sensor arrangement as claimed in the invention is made especially simple when the layer or the layers of the insulation area are made or are being made as spontaneously self-organizing layers or as self-assembling layers. In doing so advantageously the tendency and the striving of certain essentially liquid or liquid-dissolved parent substances to form an ordered and/or layer-like structure on the surface under the influence of the interaction with the structure of the surface in a spontaneous and self-organizing manner is used, which layer-like structure under certain circumstances and for certain classes of substances leads to formation of especially thin and optionally single layers or monolayers, especially of molecules.

Here it is therefore advantageous if the underlayer or the undermost region of the insulation area essentially facing the electrode is a layer of an organic thio compound. With respect to the electrical properties preferably the use of a long-chain alkane thiol and/or is recommended. A base of a C₁₈ alkane, therefore octadecane thiol, is especially preferred.

In this approach the fact is used that on certain precious metal surfaces, for example gold, silver and platinum, a covalently bonded monolayer can form on the electrode surface from an organic solution which contains the corresponding thio compound in solution, as a result of the specific interaction of the thio group with the surface atoms of the precious metal electrode; this monolayer can form hexagonally dense packing with the corresponding geometry of the thio compound, by which especially low residual conductivity of the precious metal surface can be accomplished with respect to the measurement medium which is to be provided.

On the other hand, it is provided that the upper layer or the uppermost region or surface region of the insulation area facing essentially away from the electrode is a layer of an amphiphilic organic compound, especially of a lipid, and/or the like.

Likewise the arrangement and structuring of the surface of the insulation area are induced by this procedure. The amphiphilic compounds have at least one area which is made essentially polar, so that in the measurement medium which has an especially aqueous nature a certain partial solubility arises. On the other hand, the amphiphilic organic compounds have an apolar or hydrophobic area with an arrangement which is less preferred in energy terms in an aqueous measurement medium. Preferably a layered structure is formed by this phenomenon; in this structure the polar or water-soluble areas of the amphiphilic compound are assigned to the aqueous measurement medium, conversely the apolar or hydrophobic areas of the amphiphilic organic compound are located facing away from the aqueous measurement medium. Thus a monolayer can be formed which forms especially the surface area of the electrode area. This takes place preferably in combination with a alkane thiol monolayer as the underlayer so that the insulation area is essentially at least partially a double layer of two monomolecular films or monolayers.

The sequence of two monolayers which is formed in this way has certain structural similarities to certain membrane structures which are known from biological systems so that the sequence of two monolayers which has been formed in this way—specifically the alkane thiol monolayer facing the electrode and the overlying lipid monolayer—can be assigned a certain membrane structure. Based on the underlying solid carrier this membrane structure is also called a solid supported membrane (SSM). This membrane structure SSM with respect to the arrangement and property of the sensor electrode means as claimed in the invention as a capacitively coupled electrode has especially favorable properties.

In particular that area which is defined by the region of the insulation area which covers and/or insulates the electrode advantageously has the just described membrane structure. Here it is advantageous that this membrane structure has at least in part a specific electrical conductivity of roughly Gₘ≈1-100 nS/cm². Furthermore advantageously the specific electrical capacitance is roughly Cₘ≈10-1000 nF/cm². Finally, alternatively or in addition the area for the membrane structure is roughly A≈0.1-50 mm².

In another advantageous embodiment of the sensor arrangement as claimed in the invention it is provided that the region of the insulation area which covers and insulates the electrode has a membrane structure (SSM) with an area of roughly A≈0.1-50 mm² and with a specific electrical conductivity of roughly Gₘ≈1-100 nS/cm² and/or with a specific electrical capacitance of roughly Cₘ≈10-1000 nF/cm².

The high specific capacitance Cm is especially advantageous with respect to the amperometric testing of the active ingredient which is to be carried out and in which the initiated electrical actions of the essentially biological units are measured as electrical currents, specifically as displacement currents or capacitive currents.

With respect to the signal/noise ratio a corresponding sealing resistor in the range of a few nanosiemens is especially advantageous.

This can be achieved according to another embodiment of the sensor electrode means as claimed in the invention also by applying a teflon layer, for example directly to the metal electrode. Such a process is entirely sufficient for example for potentiometric tests of active ingredients since what matters here is not the high electrical capacitance, but the high sealing resistance due to the voltage measurement.

Especially simple geometrical conditions arise, in particular with respect to the reproducibility of the measurement results when the carrier, the electrode and/or the insulation area and/or their surface or boundary layer areas are made at least in part essentially planar, especially also on the microscopic plane or scale. The planarity ensures that certain field intensity effects on the edges or tips which can lead to breakdown of the sealing resistor are precluded. Furthermore, with regard to the exchange of the measurement medium which is to be provided, in operation the advantage of a homogenous boundary surface distribution arises. Possible protuberances or cavities on the boundary surface between the insulation area and the measurement medium would lead to concentration inhomogeneities which under certain circumstances could have an adverse effect on the detection or measurement results which have been obtained. The planarity, especially of the metallic boundary surfaces, can be ensured by the corresponding production processes, for example by epitactic growth, annealing or the like.

For making external contact with the sensor electrode means, for example with an external measurement circuit or the like, there is a contact area, especially the corresponding insulation being formed to prevent other short circuits, especially with regard to the measurement medium.

It is especially advantageous with respect to the high throughput in a test of the active site and/or active ingredient which is to be carried out accordingly if the sensor electrode means as claimed in the invention is made such that at least in operation it has essentially constant mechanical, electrical and/or structural properties relative to liquid flows with high flow velocities, preferably in the range of roughly v≈0.1-2 m/s, especially in the area of the membrane structure and/or especially with respect to the attachment and/or arrangement of primary carriers. This required and advantageous constancy of the mechanical, electrical and/or structural properties of the sensor electrode means as claimed in the invention and especially the membrane structure provided in it result inherently from the aforementioned measure for formation of the electrode and the insulating layers which cover the electrode, especially in the form of self-assembling monolayers of an alkane thiol on gold with a corresponding monolayer of lipid in an aqueous medium.

Advantageously the sensor arrangement as claimed in the invention with the described sensor means is used in a process for pharmacological testing of an active site and/or active ingredient using amperometric and/or potentiometric means and in a device for carrying out this process.

In the sensor arrangement as claimed in the invention the primary carrier can be a eukaryotic cell, a procaryotic cell, organelles thereof, a bacterial unit, a viral unit, and/or the like and/or components, fragments, especially membrane fragments or the like, and/or associations thereof in essentially native and/or in altered, especially purified, microbiologically and/or molecular biologically altered form.

It is thus fundamentally conceivable for isolated and entire cells to be used as primary carriers of the corresponding biological units which can be activated into an electrical action, whether of plant or animal origin. Thus for example the study of entire cardiac cells is possible and conceivable. On the other hand the examination of plant cells, for example algae cells or other single cells, can be imagined. In addition, certain bacteria or viruses as a whole can be studied. Furthermore, it is conceivable by certain microbiological or biochemical measures to use components or fragments of cells, bacteria or viruses as the primary carriers. Furthermore it is also conceivable to use associations of cells, bacteria or the like as the primary carriers and to couple them to the corresponding sensor electrode means for forming a sensor arrangement as claimed in the invention.

Furthermore as claimed in the invention it is possible to use all of these proposed primary carriers in their native form or in an altered form. In doing so for example eukaryotic cells, procaryotic cells or bacteria can be used which have been altered by the corresponding purification, microbiological and/or molecular biological processes, for example to preferably form certain proteins with certain desired properties.

In addition to the primary carriers which are already available in natural form, in the form of cells, bacteria, and the like, it is also conceivable to produce artificial primary carriers, for example in the form of vesicles, liposomes, micellar structures and/or the like. They are then optionally provided and/or enriched with the corresponding biological units which can be activated to electrical action. The corresponding processes for reconstitution of membrane proteins or the like in vesicles or liposomes are known and can be used here in an advantageous manner to create especially advantageous embodiments of the sensor arrangement as claimed in the invention.

As essentially biological units all units are possible which can be induced to an at least partially electrically formed action. In particular, those biological units are conceivable which can be activated at least to partially electrogenic and or electrophoretic charge carrier transport and/or at least to partially electrogenic and/or electrophoretic charge carrier movement and which constitute biological, chemical and biochemical units. They are especially transport units which move charge carriers upon their activation. Components, fragments and/or associations of these units, especially transport units, are also conceivable.

In particular as biological units membrane proteins are recommended, especially ion pumps, ion channels, transporters, receptors and/or the like. With reference to many of these biological units there are findings and/or assumptions regarding the fact that certain processes are associated with at least one electrogenic component step. These electrogenic component steps can be associated with actual transport of substances, as for example in a channel, an ion pump, or certain transporters. But biological units, especially membrane proteins, are also known with electrical activity which is not associated with net transport of substances, but simply with optionally reversible charge displacement within the framework of a conformation change or bonding or the like. These electrical activities can also be fundamentally measured as claimed in the invention as brief displacement currents and/or potential changes.

The biological units, especially the membrane proteins, can be provided in essentially native form and/or in an altered, especially purified, microbiologically and/or molecular biologically altered form. On the one hand, certain native properties can be tested for example in the organism of existing proteins and pharmacologically studied. On the other hand changes initiated by molecular biology or genetic engineering are also recommended to analyze certain aspects, for example of the transport or the pharmacological mode of action of an active ingredient.

It is especially advantageous for there to be primary carriers of an essentially standard type of primary carriers. This is important with respect to the meaning and analysis of a test of an active ingredient which is as unequivocal as possible and relates to the geometrical, physical, chemical, biological and molecular biological properties of the primary carriers.

This also applies to the biological units provided in the primary carrier, especially for the membrane proteins or the like. Here biological units of an essentially standard type are provided, especially with respect to their geometrical, physical, chemical, biological and molecular biological properties. In addition the biological units will be advantageously roughly standard with respect to their orientation and/or with respect to their activation capacity relative to the respective primary carrier.

To achieve a signal quality as high as possible it is advantageous that the surfaces of the primary carriers and/or secondary carriers are made such that attachment and/or placement of the primary carrier on the secondary carrier is promoted. This yields on the one hand an especially large number of attached primary carriers and/or especially intimate contact of the primary carriers on the secondary carrier, by which the electrical coupling and thus the signal/noise ratio are increased.

The attachment can be controlled for example via the so-called lipid-lipid interaction between the primary carrier, for example a vesicle, and the secondary carrier, for example lipid-thiol SSM. On the other hand, a covalent bond of the primary carrier on the surface of the secondary carrier is conceivable, for example in the form of a biotin-streptavidin pattern or in the sense of a his-tag coupling.

Here it is especially advantageous if the surfaces of the primary carrier and the secondary carrier are made polarly opposite one another. The promotes the attachment rate of the primary carrier on the secondary carrier and the strength of the contact between them.

It is especially advantageous if the primary carriers are essentially vesicles or liposomes of the same type and/or of the same action, preferably from a lipid, in and/or on their membrane units of essentially one type of membrane proteins being inserted and/or attached preferably in essentially oriented form.

The sensor arrangement as claimed in the invention is advantageously used by a process for amperometric and/or potentiometric, especially pharmacological testing of an active site and/or active ingredient and/or in a device for carrying out such a process.

The device as claimed in the invention for pharmacological testing of an active site and/or active ingredient using amperometric and/or potentiometric means has at least one measurement area. Furthermore, the measurement probe for receiving and/or forming the measurement signals is at sensor arrangement as claimed in the invention. Furthermore, there is a data acquisition/control means which is made at least for detecting the measurement signals of the respective measurement probe. It is essential to provide an exchange/mixing means which is designed for making available, exchanging, mixing and/or adjusting of the measurement medium. The sensor arrangement is located in the measurement area as the measurement space or measurement site.

Such a device can be built for example in the form of a so-called patchclamp apparatus in which an electrolyte bath constitutes the actual measurement area. The measurement probe would conventionally be a so-called patch electrode or patch pipette to which the biological unit to be studied, for example a cell or the like, is suctioned and held. Here the above described sensor arrangement is formed as claimed in the invention. The data acquisition/control means is formed by the entire measurement electronics, corresponding micromanipulators and by the control electronics for building up and mixing the electrolyte bath. The exchange/mixing means can consist for example of the corresponding tube systems with perfusors or the like connected to them.

The device as claimed in the invention for pharmacological testing of an active site and/or active ingredient using amperometric and/or potentiometric means therefore has the above described sensor arrangement as claimed in the invention as the measurement probe. This advantageously yields an especially stable and durable measurement arrangement with which for a time interval which has been greatly prolonged compared to the conventional approach a host of test runs under the most varied test conditions can be carried out without adverse effects on the detection accuracy, signal quality or other properties of the measurement probe. Furthermore, as a result of the durability of the measurement probe a higher speed can be used; this relates both to the handling of the actual sensor and also the exchange rate or flow velocity of the fluid measurement medium in the measurement area. Conventional patchclamp and/or voltage clamp arrangements are a problem in contrast thereto, as is generally known, with respect to the reproducibility of the measurement results and the stability of the measurement system.

Advantageously, especially via the exchange means, via the measurement medium the measurement conditions, especially the pharmacological conditions, according to another embodiment of the device as claimed in the invention for pharmacological testing, can be adjusted and/or changed in a defined manner, especially continuously, preferably in the manner of a continuous flow system.

In contrast to the prior art in which adjustability and/or alterability of the measurement conditions defined in this way is possible only with difficulty or at increased cost, due to the stability of the measurement probe used as claimed in the invention exchange and/or alteration of the measurement medium is easily possible. Also the corresponding changes with respect to substrate conditions or other properties of the measurement environment can be easily achieved within a short time by exchange.

In particular it is a good idea to use as the exchange/mixing means a pump system, perfusor system and/or the like in order to form a measurement arrangement in which flow of the measurement medium takes place past the measurement probe continuously within the framework of the flow system. In such a flow system then the corresponding measurement conditions to be studied can be formed by external admixture.

Here it is especially advantageous that in the measurement area flow velocities of roughly v≈0.1-2 m/s can be produced, especially in the area, immediate vicinity or the neighborhood of the measurement probe and/or especially by the provided exchange/mixing means.

The advantage of high flow velocities arises on the one hand from the mechanical stability of the sensor arrangement as claimed in the invention and the sensor electrode means underlying it.

In addition it is however especially advantageous that in addition to the use of entire cells, bacteria, or the like which are made comparatively large, advantageously also vesicles and liposomes as well as membrane fragments can be used as the primary carriers for the biological, especially membrane proteins. Vesicles, liposomes, membrane fragments and the like are made comparatively small and compared to their size or their surface have a comparatively stronger tendency to attachment or adsorption on the surface of the measurement probe. In addition, due to their smaller surface in the area of the flow of the measurement medium compared to entire cells or the like they experience very much smaller shear forces so that even at higher flow velocities they remain adhering to the sensor electrode means as claimed in the invention within the framework of the sensor arrangement as claimed in the invention so that the experimental or test conditions do not change.

Advantageously the measurement area is made simply as an essentially closed vessel or vessel means. Here according to another embodiment of the device as claimed in the invention it is provided that the corresponding feed and/or drain means be made for flow connection to the exchange/mixing device. The measurement area can be made for example as a type of compartment or cell, with the measurement probe in the form of a sensor arrangement made as claimed in the invention located on its bottom area.

In another advantageous embodiment of the device as claimed in the invention for pharmacological testing of an active site and/or active ingredient using amperometric and/or potentiometric means there is a plurality of integrated sensor arrangements, preferably in separate and independent recess areas of a microplate or microtiter plate, which areas have been decoupled from one another in terms of electricity and flow, preferably in order to form 8, 12, 96 measurement channels on a grid.

It is also conceivable that there is a plurality of measurement probes, especially to simultaneously carry out parallel operation for several independent test series. Then it is furthermore advantageous for the data acquisition/control means to be made to receive and/or detect measurement signals from a plurality of measurement probes independently and/or decoupled from one another. This ensures that the different measurement probes and their measurement signals can be evaluated separately from one another and can be examined with greater accuracy and thus enables implementation of simultaneous experiments or test processes which are independent of one another, and especially a higher test throughput with lower costs per measurement can be achieved.

Moreover it is then advantageous for the exchange/mixing means and/or the measurement area to be made in order to adjust and/or change in a defined manner for the plurality of the measurement probes the corresponding measurement conditions independently and/or decoupled from one another. The measurement area can be for example an arrangement of cells or compartments separated from one another in terms of flow. Here there can also be a common exchange/measurement means via which for all measurement areas the same measurement conditions, for example with respect to the measurement medium or the like, are simultaneously formed. But flow decoupling and especially electrical decoupling of the measurement areas which are to be examined independently of one another are important here.

In particular it is however advantageous to devise a measurement arrangement for the device as claimed in the invention in which the test processes on the measurement sensors can be carried out in more or less miniaturized form. For example, the measurement area can be made in the form of or in the grid of a microplate or microtiter plate or the like, a plurality of the integrated measurement probes being made preferably in separate recess areas thereof which are independent of one another and/or are decoupled from one another in terms of flow and/or electricity. Preferably the recess areas and the measurement probes in them can be arranged in the form of a grid on such a microplate in order to implement an arrangement of 4, 8, 12, 96 or the like parallel measurement channels. In doing so accordingly current 4, 8, 12, 96-channel automatic pipettes for sample addition can be used.

As was already explained, in the process as claimed in the invention for pharmacological testing of an active site and/or active ingredient using amperometric and/or potentiometric means there can be a biological unit which is to be used or studied on or in at least one measurement probe. The measurement probe for its part is located in a measurement medium, and the measurement medium for example can be an essentially aqueous medium. Furthermore, in the actual testing process via the provided measurement probe the respectively initiated electrical action of the essentially biological unit is measured, especially as an electrical voltage and/or as an electrical current.

The underlying object of the invention is achieved in terms of the process in that in the known processes the sensor electrode means detailed above, the corresponding sensor arrangement and/or the described device as claimed in the invention are used for amperometric and/or potentiometric, especially pharmacological testing of an active site and/or active ingredient.

Here it is especially advantageous for the intended measurement medium to flow around or against the measurement probe at least partially and/or at least temporarily. This process allows the test conditions to be easily changed, specifically by an external mixing or exchange process, without disruption, in contrast to the procedure in the prior art, for example in the measurement process according to patchclamp technology or voltage clamp technology.

The process as claimed in the invention is especially advantageous in that a flow velocity of the measurement medium of roughly v≈0.1-2 m/s is used because as a result of these high flow velocities kinetic studies on the biological units, especially on the membrane proteins, can also be carried out in which with high time resolution the signal development is recorded and characterized and referenced back to the individual functional steps of the biological unit.

As a result of the stability of the sensor electrode means, the sensor arrangement and/or device used in the process as claimed in the invention, advantageously it becomes possible to carry out a plurality or host of tests in succession, especially by successive exchange and/or adjustment or mixing of the measurement medium, and optionally there can be washing or flushing processes interposed. For the same biological unit, for example for a given receptor or another membrane protein, which, once incorporated in a vesicle system, was inserted on the sensor electrode means provided as claimed in the invention in the process, a host of substances which are added in a mixing process to the measurement medium can be tested by this process. If this also takes place at the same time in parallel operation, in one measurement pass a host of essentially the same tests can be carried out in order for example to obtain a corresponding statistical evaluation of the manner of action of certain active ingredients on a given biological unit.

Often it is not so much the manner of action of a certain active ingredient on a known biological unit which is of interest, but rather the manner of operation of a biological unit which is unknown in this respect, which is to be clarified. Of course a host of tests which are independent of one another can be carried out.

This is especially the case in so-called deorphaning. Here an orphan is assumed. This is a genetic information unit by which a certain biological unit of so far unknown function, for example a membrane protein or the like, is coded.

Accordingly, it is provided according to one especially preferred embodiment of the process as claimed in the invention that a deorphaning process is carried out with a plurality or a host of tests, as the biological unit a product or the like of an orphan or the like, especially one of unknown function, being used as the biological unit, especially to clarify its functionality. Accordingly it is provided as claimed in the invention that the sensor electrode means as claimed in the invention, the sensor arrangement as claimed in the invention and/or the device as claimed in the invention can be used in a deorphaning process.

This invention is detailed below using a schematic based on preferred embodiments.

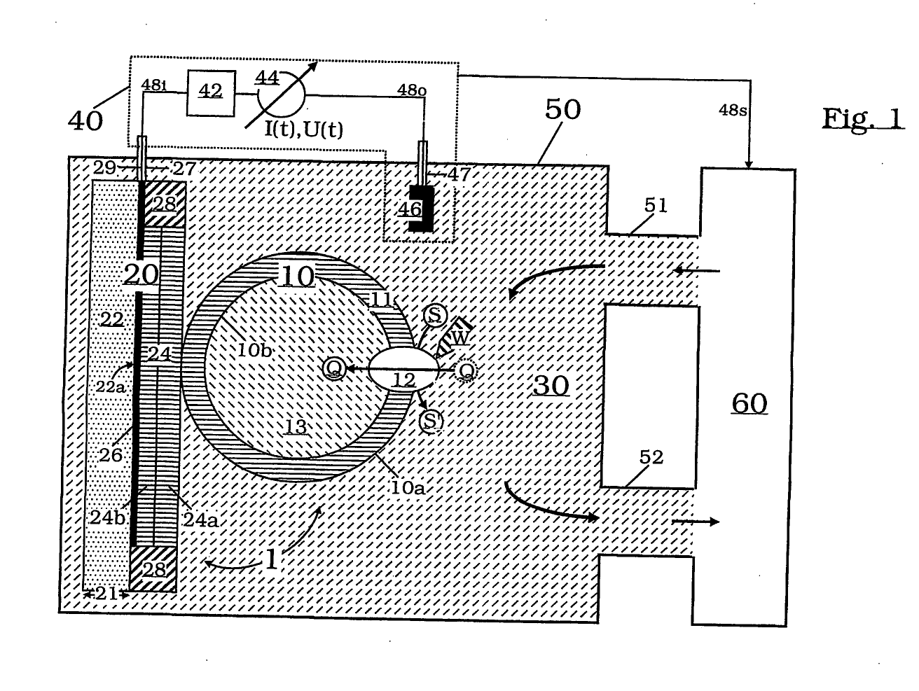

FIG. 1 shows a schematic and partially cutaway side view of one embodiment of the device as claimed in the invention using the sensor arrangement as claimed in the invention.

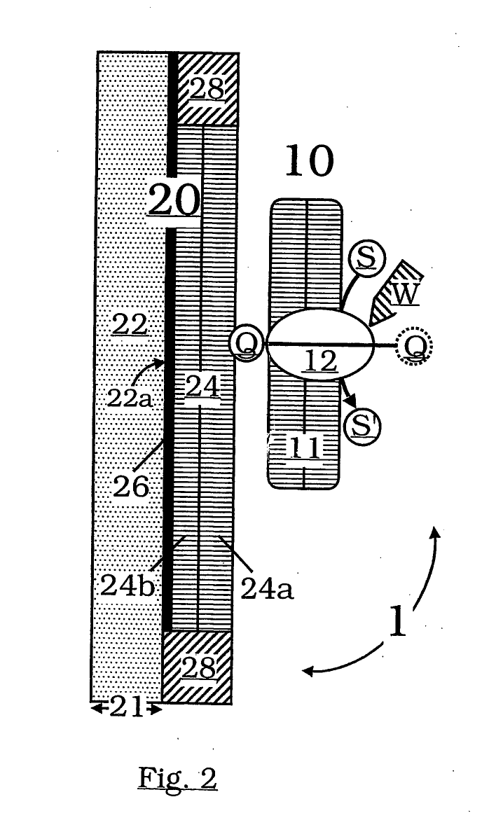

FIG. 2 shows one embodiment of a sensor arrangement as claimed in the invention with a membrane fragment as the primary carrier.

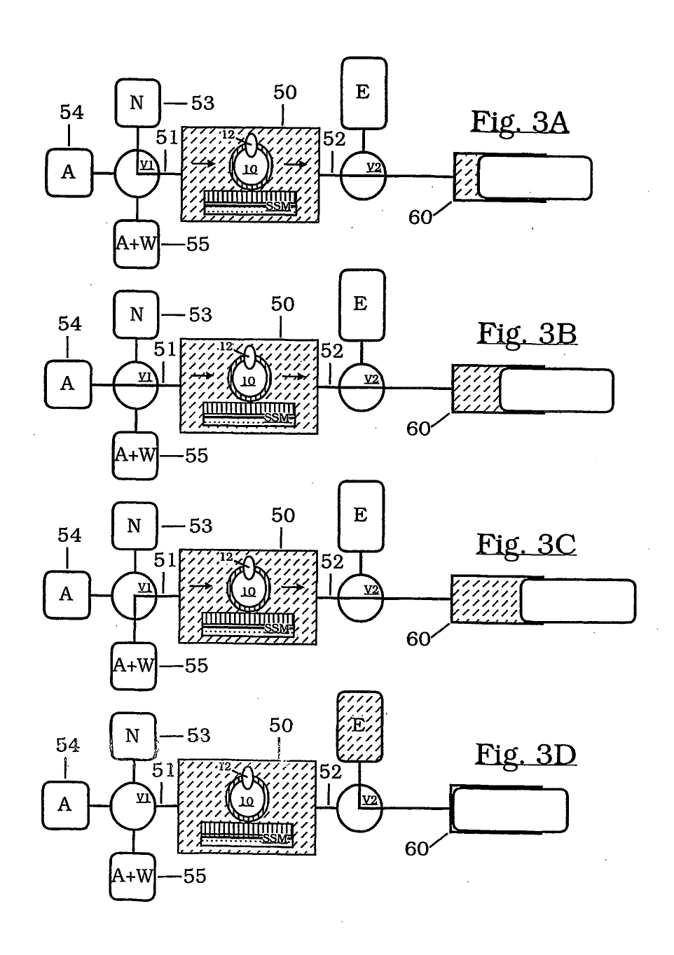

FIGS. 3A-D show in a schematic side view one embodiment of the process as claimed in the invention for pharmacological testing of an active site and/or active ingredient.

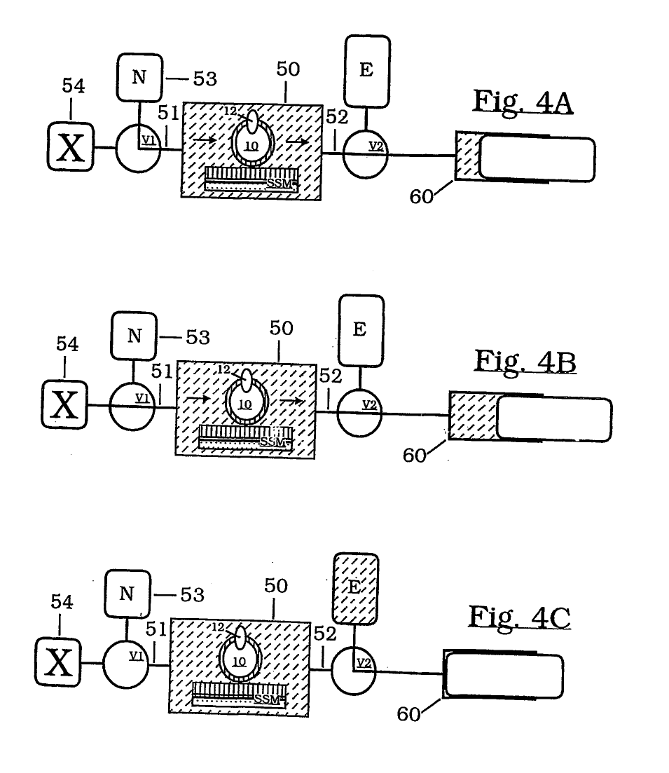

FIGS. 4A-C show in a schematic side view another embodiment of the process as claimed in the invention for pharmacological testing of an active site and/or active ingredient.

FIG. 1 shows in a schematic and partially cutaway side view of one embodiment of the device as claimed in the invention for pharmacological testing of an active ingredient.

The measurement area 50 in the form of an essentially closed vessel together with an exchange/mixing means 60, for example in the form of a perfusor system or a pump system, forms a closed liquid circuit. Communication of the liquid which is used as the measurement medium 30 takes place via the corresponding feed and drain means 51 and 52. The measurement medium 30 can be an aqueous electrolyte solution here, which has certain ion portions, a given temperature, a certain pH value, etc. Furthermore, in the measurement medium 30 optionally certain substrate substances S and/or certain active ingredients W are contained, or they are added in later process steps by the exchange/mixing means 60.

In the measurement area 50 there is a sensor arrangement 1 as claimed in the invention. The sensor arrangement 1 consists of primary carriers 10 which are attached to the surface area 24a of the sensor electrode means 20 which is used as the secondary carrier.

In the embodiment shown in FIG. 1 in schematic form and not to scale there is only a single primary carrier 10. It consists of a lipid vesicle or liposome in the form of a lipid double layer or lipid membrane 11 which is made essentially hemispherical and closed. As the essentially the biological unit 12 a membrane protein extending through the membrane is inserted into this lipid double layer 11 of the vesicle which is used as the primary carrier 10.

By conversion of a substrate S which is present in the measurement medium 30 into a converted substrate S′ certain processes are initiated in the membrane protein 12 and in the case shown in FIG. 1 lead to transport of the substances of one species Q from the extravesicular side or outside 10a of the vesicle 10 to the intravesicular side or inside 10b of the vesicle 10. If the species Q contains an electrical charge, transport of this species Q from the side 10a to the side 10b leads to net charge transport which corresponds to an electrical current from the outside 10a of the vesicle 10 to the inside 10b of the vesicle 10.

On the one hand, in each vesicle 10 generally a host of essentially identical membrane protein molecules 12 in essentially the same orientation in the membrane 11 of the vesicle 10 are incorporated. If they are activated essentially simultaneously—for example by a concentration jump which is initiated by mixing, in the concentration of the substrate S from a nonactivating measurement medium N, 30 without a substrate S to an activating measurement medium A, 30 with the substrate S, this leads to a measurable electrical current.

This charge carrier transport is therefore measurable because a host of primary carriers 10 or vesicles are attached to the surface 24a of the sensor electrode means 20 so that upon activation of a host of protein molecules 12 in a host of vesicles in front of the surface 24a of the sensor electrode means 20 a space charge of a certain polarity forms. The space charge then acts on the electrode 26 which in the case shown in FIG. 1 is vapor deposited onto the glass carrier 22 in the form of a gold layer and is covered by a double layer which is used as the insulating area 24 and which consist of a lower layer 24b and an upper layer 24a which is used as the surface and is electrically insulated relative to the measurement medium 30.

The surface or the upper layer 24a of the insulation area 24 is for example a lipid monolayer which is compatible with the lipid double layer 11 of the vesicle 10 and which is formed by means of a self-assembling process on an alkane thiol monolayer which forms the lower layer 24b, so that the sequence of layers 24b and 24a, specifically the sequence of an alkane thiol monolayer and a lipid monolayer on a gold substrate formed like a solid as the electrode 26, forms a membrane structure SSM which is also called a solid supported membrane (SSM).

Via a connecting line 48i the sensor arrangement 1 and especially the sensor electrode means 20 are connected to a data acquisition/control means 40. The latter has a measurement means 44 in which an electrical current I(t) or an electrical voltage U(t) can be measured as a function of time.

Furthermore there is an amplifier means 42 in which the measurement signals are filtered and/or amplified. Via a control line 48s the testing of the active ingredient is controlled by controlling the exchange/mixing means 60. Via another line 48o the electrical circuit is closed by means of an opposing electrode 46, for example in the form of a Pt/Pt electrode or by means of an Ag/AgCl electrode. Insulation 28, 27 and 47 prevents short circuits of the SSM or the opposing electrode 46 relative to the measurement medium 30.

FIG. 2 shows in a schematic and partially cutaway side view one embodiment of the sensor arrangement 1 as claimed in the invention in which instead of a vesicle or liposome the primary carrier 10 is a membrane fragment into which a membrane protein which is used as the biological unit 12 is inserted in an oriented manner. With reference to the embodiment of FIG. 2 it can also be recorded that the figure is not to scale, and on the other hand generally a large plurality of membrane fragments are attached or adsorbed at the same time on the SSM or the surface 24a of the sensor electrode means 20 which is used as the secondary carrier.

Here it is also shown again that by conversion of the substrate S which is provided in the measurement medium 30 into a converted substrate S′ the transport of the substance of the species Q from one side 10a of the membrane fragment 10 to the opposing side 10b takes place and can be detected as a function of time via the corresponding net charge transport and the associated displacement current.

FIGS. 3A to 3D show one embodiment of the process as claimed in the invention for pharmacological testing of an active site and/or active ingredient using amperometric and/or potentiometric means.

In a measurement area 50 which is made as a cell, on one SSM which is used as the sensor electrode means 20 an assembly of vesicles 10 with a membrane protein 12 which is inserted there is adsorbed. The sensor arrangement 1 which has been made in this way is incubated in the measurement area 50 in the provided measurement medium 30.

The measurement area 50 is connected over a feed line 51 via a first valve means V1 in a controllable manner to a plurality of the storage vessels 53, 54 and 55 in which there are certain volumes of the measurement medium 30 with certain additional ingredients. Thus the first storage vessel 53 of FIGS. 3A to 3D contains a nonactivating measurement medium N which is chosen such that the membrane proteins 12 inserted into the vesicles 10 are not initiated into electrical action by the composition of the measurement medium of type N. The second storage vessel 54 of FIGS. 3A to 3D contains a measurement medium A by which the membrane proteins 12 in the vesicles 10 can be activated to an electrical action, therefore to charge transport or the like. In the third storage vessel 55 of FIGS. 3A to 3D finally an active ingredient W has been added to the activating measurement medium A of the vessel 54 with an effect on the activity of the protein 12 in the vesicles 10 which is to be determined.

Via a drain means 52 or a drain line the measurement area 50 or the cell is connected via the second valve means V2 at least to part of the exchange/mixing means 60. Furthermore, via the valve V2 a disposal vessel E can be connected for example in order to empty the exchange/mixing means 60.

In the first phase of the pharmacological testing of an active ingredient which is shown in FIG. 3A, the measurement area 50 is connected to the first storage vessel 53 via the first valve means V1 and the supply line 51. On the other hand, the measurement area 50 is connected via the drain means 52 and via the second valve means V2 to the exchange/mixing means 60. By operating the exchange/mixing means 60 a suction force is exerted via the areas which are connected to one another so that the nonactivating measurement medium N flows out of the storage vessel 53 through the cell of the measurement area 50 and the SSM and flows around or flushes the protein-containing vesicles 10. In this state the electrical activity of the protein 12 cannot be measured and this phase is used for equilibration of the SSM and to record noise signals and to balance noise.

In the second test phase shown in FIG. 3B, via the data acquisition/control means 40 the first valve means V1 is connected such that the supply means 51 of the measurement area 50 is connected to communicate with the second storage vessel 54 so that the activating measurement medium A flows through and flushes the cell of the measurement area 50, the SSM and the protein-containing vesicles as a result of operation of the exchange/mixing means 60, and thus excites the membrane proteins 12 contained in the vesicles 10 to electrogenic charge transport which can then be detected via the data acquisition which is not shown in FIGS. 3A-3D.

In the third phase of the process of pharmacological testing of an active ingredient which is shown in FIG. 3C the first valve means V1 is connected such that the measurement area 50 is flow-connected to the third storage vessel 55 so that as a result of operation of the exchange/mixing means 60 the activating measurement medium A with the addition of the active ingredient now flows through the cell of the measurement area 50 and thus flushes the SSM and the protein-containing vesicles 10.

By recording possible electrical signals under the influence of the added active ingredient W, compared to the signals which have been recorded during the phase of FIG. 3B the effect of the active ingredient on the activity of the protein 12 in the vesicles can be essentially determined. In the phase which is shown in FIG. 3D the amount of liquid held in the exchange/mixing means 60 is disposed of in the disposal vessel E, by the second valve means V2 the exchange/mixing means 60 being connected exclusively to the disposal vessel E.

Of course the arrangement shown in FIGS. 3A to 3D and the associated test process can also be more complex and can contain additional intermediate measurement steps, flushing and washing processes.

The basic advantages of this invention are the high mechanical stability of the sensor electrode means provided and the sensor arrangement and with that high operating readiness, ease of handling and low fault susceptibility. In the use of the sensor electrode means as claimed in the invention and the sensor arrangement as claimed in the invention within the framework of the pharmacological test of the active ingredient, there arise a long service life, high reliability, low fault susceptibility and especially a test throughput which is greatly increased compared to conventional processes, by which the corresponding test processes can be economically planned and carried out.

FIGS. 4A to 4C describe another embodiment of the process as claimed in the invention. But here there are only two storage vessels 53 and 54. The storage vessel 53 contains again a nonactivating solution N. The storage vessel 54 contains as X either in a first process step the activating solution A and in the second process step an activating solution A+W with the active ingredient W which is to be tested. In the former case X=A the activation of the biological unit is measured as the reference signal. In the latter case X=A+W the effect of the active ingredient on activation becomes measurable.

In the simplest case the active agent W can be identical to S and/or the transport species Q.

### REFERENCE NUMBER LIST

1 sensor arrangement
10 primary carrier, vesicle, membrane fragment
10a surface, outside, extravesicular side
10b inside, intravesicular side
11 membrane
12 biological unit, membrane protein
13 inner medium
20 sensor electrode means
21 electrode area
22 carrier
22a surface region
24 insulation area
24a upper layer, surface region, lipid monolayer
24b underlayer, thiol/mercaptan monolayer
26 electrode
27 insulation
28 insulation
29 terminal
30 measurement medium
40 data acquisition/control means
42 amplifier means
44 measurement means
46 opposing electrode
48i,o connecting lines
48s control line
50 measurement area, vessel, cell
51 supply means
52 drain means
53 storage vessel
54 storage vessel
55 storage vessel
60 exchange/mixing means
100 measurement device
A activating measurement medium
A+W activating measurement medium with active ingredient W
Cₘ specific capacitance
E disposal
Gₘ specific conductivity
N nonactivating measurement medium
I(t) current signal
Q charge carrier
S substrate
S′ converted substrate
SSM membrane structure, solid support membrane
U(t) voltage signal
V flow velocity
V1 first valve means
V2 second valve means
W active ingredient

1-17. (canceled)

18. A device for pharmacological testing of an active site and/or active ingredient using amperometric and/or potentiometric means, comprising:
at least a measurement area which comprises a measurement probe, said measurement probe comprising a plurality of sensor arrangements;
a data acquisition/control device for acquiring measurement data from the sensor arrangement; and
an exchange and mixing means for making available, exchanging, mixing and/or adjusting the measurement medium,
wherein the sensor arrangements are integrated, and wherein each of said sensor arrangements comprises:
a secondary carrier comprising an electrically conductive and solid electrode area;
a plurality of primary carriers located in the immediate spatial vicinity of the secondary carrier, each of the primary carriers comprising a biological unit which can be activated into electrical action; and
an aqueous measurement medium containing the secondary carrier and primary carriers,
wherein each electrode area is electrically insulated relative to (a) the measurement medium, (b) the primary carriers and (c) the biological unit,
wherein each electrode area comprises at least one electrically conductive electrode electrically insulated by an insulation area from the measurement medium, the primary carriers and the biological units,
wherein each insulation area comprises at least a sequence of spontaneously self-organizing monolayers,
wherein the monolayers comprise an upper layer and under layer, the upper layer facing away from the electrode and the under layer facing the electrode, the upper layer comprising a layer of an amphiphilic organic compound, and the under layer comprising a layer of an organic thio compound, and
wherein the amphiphilic organic compound is a lipid, and the organic thio compound is a long-chain alkyl thiol.

19. The device according to claim 18, wherein the sensor arrangements are in separate and independent recess areas of a microplate or microtiter plate, which areas have been decoupled from one another in terms of electricity and flow.

20. The device according to claim 19, wherein the sensor arrangements form 8, 12, 96 measurement channels on a grid.

21. The device according to claim 18, wherein measurement conditions can be adjusted in a defined manner with the exchange and mixing means via the measurement medium, and wherein a flow velocity of roughly 0.1-2 m/s can be produced by the exchange and mixing means in the vicinity of the sensor arrangement.

22. The device according to claim 21, wherein the measurement conditions are pharmacological conditions, and wherein the pharmacological conditions are adjusted in the manner of a continuous flow system.

23. The device according to claim 18, wherein the primary carriers comprise eukaryotic cells, prokaryotic cells, viruses, vesicles, liposomes or micellar structures, or wherein the primary carriers comprise components or associations of a eukaryotic cell, prokaryotic cell or virus in native or altered form.

24. The device according to claim 23, wherein the prokaryotic cells are bacteria, and the prokaryotic cell is a bacterium.

25. The device according to claim 23, wherein the primary carriers are membrane fragments of a eukaryotic cell or prokaryotic cell.

26. The device according to claim 23, wherein the biological unit comprises a membrane protein.

27. The device according to claim 18, wherein the long-chain alkyl thiol is octadecyl thiol.

28. The device according to claim 18, wherein a region of the insulation area covers and insulates the electrode, said region comprising a membrane structure with an area of about 0.1-50 mm² and with a specific electrical conductivity of about 1-100 nS/cm² and/or with a specific electrical capacitance of about 10-1000 nF/cm².

29. The device according to claim 18, wherein the electrode comprises a metallic material or an electrically conductive metal oxide.

30. The device according to claim 29, wherein the metallic material is a precious metal and the electrically conductive metal oxide is indium tin oxide.

31. The device according to claim 30, wherein the precious metal is gold, platinum or silver.

32. The device according to claim 18, wherein the biological unit is activated by electrogenic charge carrier movement.

33. The device according to claim 32, wherein the electrogenic charge carrier movement is a electrogenic charge carrier transport.

34. The device according to claim 26, wherein the biological unit comprises an ion pump, ion channel, transporter, receptor, or a component or an association thereof.

35. The device according to claim 18, wherein the biological unit is in a native form.

36. The device according to claim 18, wherein the biological unit is in a form altered from a native form.

37. The device according to claim 36, wherein the biological unit is in a puffered form microbiologically and/or molecular biologically altered from the native form.

38. The device according to claim 18, wherein a surface of a primary carrier and a surface of a secondary carrier are of opposite polarity or oppositely charged, and/or wherein a surface of a primary carrier and a surface of a secondary carrier are attached via a chemical bond.

39. The device according to claim 38, wherein the surface of the primary carrier and the surface of the secondary carrier are attached via his-tag coupling or streptavidin-biotin coupling.
